(19) United States
(12) Patent Application Publication
(10) Pub. No.: US 2023/0392205 A1
(43) Pub. Date: Dec. 7, 2023

(54) L-DNA ENCRYPTED TRACERS

(71) Applicant: Vanderbilt University, Nashville, TN (US)

(72) Inventors: Nicholas M. ADAMS, Nashville, TN (US); Frederick R. HASELTON, Nashville, TN (US)

(73) Assignee: Vanderbilt University, Nashville, TN (US)

(21) Appl. No.: 18/324,828

(22) Filed: May 26, 2023

Related U.S. Application Data
(60) Provisional application No. 63/346,625, filed on May 27, 2022.
(60) Provisional application No. 63/413,061, filed on Oct. 4, 2022.

(51) Int. Cl. C12Q 1/6876; C12Q 1/6818

(52) U.S. Cl. CPC C12Q 1/6876; C12Q 1/6818

(57) ABSTRACT

The present disclosure is directed to the use of left-handed DNA (L-DNA) tracer to identify the source, track the distribution, and validate the integrity of products or resources that are highly regulated, valuable, or hazardous (e.g., pharmaceuticals, treated water, chemicals, designer products, and ammunitions). L-DNA tracers can encrypt unique identifying information, as well as more general information about the type of product, such as the manufacturing location, source, and date, directly into the nucleotide sequence. The L-DNA tracers can embed directly into the product so that it could neither be disassociated from the product nor be re-associated with another product. Because there are no technologies available to sequence L-DNA, the L-DNA tracers cannot be reverse engineered, copied, or falsified. The L-DNA tracers are only deciphered using a unique detection key. L-DNA tracers therefore can be used to preserve the integrity of product supply chains, allow counterfeit products to be detected, and identify the source of highly regulated products.

### PRIORITY CLAIM

This application claims benefit of priority to U.S. Provisional Application Ser. Nos. 63/346,625 and 63/413,061, filed May 27, 2022, and Oct. 4, 2022, respectively, the entire contents of which are hereby incorporated by reference.

### STATEMENT REGARDING FEDERALLY FUNDED RESEARCH

This invention was made with government support under grant no. R42HG009470, awarded by the National Institutes of Health. The government has certain rights in the invention.

### INCORPORATION OF A SEQUENCE LISTING

This application contains a Sequence Listing XML, which has been submitted electronically and is hereby incorporated by reference in its entirety. Said XML Sequence Listing, created on May 24, 2023, is named VBLTP0306US.xml and is 1,868 bytes in size.

### BACKGROUND

### 1. Field of the Disclosure

The present disclosure relates generally to fields of biology, chemistry, manufacturing and forensics. More particular, the disclosure relates to the design, synthesis and use of L-DNA tracer molecules to mark goods and products.

### 2. Background

Many manufactured products benefit from the use of labeling to establish the origin, authenticity, distribution, and/or appropriate use of highly regulated, valuable, or hazardous products or resources. There are hundreds of such potential applications across many industries, including pharmaceutical tracking, drug compliance urine testing, currency authentication, treated water validation, upscale food or drink authentication, pesticides tracking, chemical tracking, designer product authentication, ammunition tracking, and explosive tracking.

In one particular aspect, this relates to the governments and regulatory agencies around the world are currently improving their policies related to distribution of pharmaceuticals by creating regulations and legislation that require tracking systems to be put in place to ensure the quality and integrity of pharmaceutical supply chains. Since 2007, millions of doses of falsified medicines for all kinds of illnesses, including cancer, heart disease, asthma, and mental health disorders, have been reported to enter the United States and European supply chains (world-wide-web at pharmaceutical-technology.com/features/feature-track-and-trace-new-era-compliance-drug-manufacturers/). To prevent the introduction of counterfeit prescriptions from entering into the supply, in 2013 the United States government passed the Drug Supply Chain Security Act (DSCSA), which progressively introduces regulations from 2015 through 2023 that require pharmaceutical companies to implement a tracking system, so that the source and integrity of the drugs can be tracked through the distribution chain. In February 2019, the European Union rolled out a very similar program called the Falsified Medicines Directive (FMD). These policies mandate that 2D barcodes are printed and stuck to the external packaging of pharmaceutical products and that these barcodes are scanned and logged at each point along the supply chain until they are handed off to their intended patients.

As such, improved materials and process for marking and tracking such products are urgently needed.

### SUMMARY

Thus, in accordance with the present disclosure, there is provided a nucleic acid comprising a first region that is an L-DNA segment having a randomized contiguous sequence that is a unique identifier for the nucleic acid molecule. The nucleic acid may further comprise (a) a second region comprising a non-randomized contiguous nucleic acid sequence that is correlated with at least one piece of data or information associated with said product; and/or (b) a third region comprising a capture moiety.

The first region may be at least 10 nucleotides in length, at least 15 nucleotides in length, at least 20 nucleotides in length, at least 25 nucleotides in length, or 10 to 30 nucleotides in length. The second region may be at least 5 nucleotides in length, at least 10 nucleotides in length, at least nucleotides in length, at least 20 nucleotides in length, at least 25 nucleotides in length, or 10 to 30 nucleotides in length. The third region may be at least 5 nucleotides in length, at least 10 nucleotides in length, at least 15 nucleotides in length, at least 20 nucleotides in length, at least 25 nucleotides in length, or 10 to 30 nucleotides in length. The nucleic acid may be about 10 to 150 nucleotides in length, about 20 to 120 nucleotides in length, about 20 to 100 nucleotides in length, about 25 to 75 nucleotides in length, 15 to 50 nucleotides in length, 15 to 30 nucleotides in length, to 20 nucleotides in length, or about 30 to 50 nucleotides in length.

The at least one piece of data or information may comprise a date of manufacture, such as for an associated product, a location of manufacture, such as for an associated product, a manufacturer, such as for an associated product, a lot number, such as for an associated product, an expiration date, such as for an associated product. The second region may comprise or consist of an L-DNA segment, a D-DNA segment, or is a chimeric L-DNA/D-DNA segment. The third region may comprise or consist of an L-DNA segment, a D-DNA segment, or is a chimeric L-DNA/D-DNA segment. The first and second regions may be L-DNA segments and are separated by a first D-DNA spacer. The first, second and third regions may be L-DNA segments and said first and second regions are separated by the first DNA spacer and said second and third regions are separated by a second DNA spacer. The first and/or second DNA spacers may be substrates for a nuclease. The third region may comprise a capture sequence. The nucleic acid may comprise a quenchable or activatable/inducible signaling agent. The first, second and optionally third sequence may be present in any order.

Also provided is a method of labeling a product comprising embedding in or attaching to a product a nucleic acid a described above, also referred to as a “tracer”, comprising a first region that is an L-DNA segment having a randomized contiguous sequence that is a unique identifier for the nucleic acid molecule. The product may be a chemical or biological composition. The chemical composition may be a pharmaceutical grade chemical composition, a chemical herbicide, a chemical pesticide, a toxic or biohazard composition. The biological composition may be a pharmaceutical-grade biological composition, biological herbicide, a biological pesticide, or a biological warfare composition. The product may be an article of manufacture, such as a pill or tablet, an explosive, a foodstuff or food additive, a cosmetic, a fuel, currency, a passport or other legal document, an electronic device, a computer chip, or a vaccine.

The method may use more than one unique tracer may be included in the product, such as 2, 3, 4, 5, 6, 7, 8, 9, 10 or more unique tracers. The more than one tracer may be present in equimolar amounts. The more than one tracer may be present in different molar amounts. The method may further comprise assembling a database that correlates said tracer with a labeled product.

In another embodiment, there is provided a method of identifying a product or sample comprising contacting an L-DNA “key” molecule with said product and determining hybridization of said L-DNA “key” to a complementary L-DNA tracer in said product, the present of said complementary L-DNA “label” being correlated with one or more identifying features for said product. The product may be a chemical or biological composition, such as wherein the chemical composition is a pharmaceutical grade chemical composition, a chemical herbicide, a chemical pesticide, a toxic or biohazard composition, or wherein the biological composition is a pharmaceutical-grade biological composition, biological herbicide, a biological pesticide, or a biological warfare composition. The product may be an article of manufacture, such as wherein the article of manufacture is a pill or tablet, an explosive, a foodstuff or food additive, a cosmetic, a fuel, currency, a passport or other legal document, an electronic device, a computer chip, or a vaccine. The sample may be obtained from a living organism, such as a mammal or a plant.

The L-DNA “key” may be an L-DNA, an L-DNA/D-DNA chimera, or an enzymatic L-DNA that is complementary to said first region. The L-DNA “key” and/or said L-DNA tracer may comprise a detectable label or label that generates a detectable signal or product after binding to the first region. The detectable label may be a chemiluminescent label, a fluorescent label, a dye, a FRET moiety, a probe target, a molecular beacon, a primer target sequence, or a capture target sequence or the detectable product is a detectable nucleic acid amplification product. The tracer may comprise a capture moiety, and said method comprises contacting said product with a capture partner that is conjugated to a substrate, such as a bead, a tube, a slide or a plate, thereby sequestering the nucleic acid onto said substrate. Determining hybridization may comprise molecular beacon detection, strand hybridization fluorescence quenching detection, aptamer-antibody detection, L-DNA array detection, mirror image restriction enzyme detection, non-enzymatic L-DNA amplification detection, L-DNA amplification by L-polymerase and L-dNTPs, or L-DNA-zyme detection. The method may further comprise reviewing a data base that correlates said L-DNA “label” with a labeled product.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. Molecular beacon detection. In this design, the encryption reagent is a specific sequence of L-DNA embedded in the test material during manufacture. During material testing the embedded material is retrieved from the material and mixed with an L-DNA molecular beacon. As shown, the decryption reagent is a molecular beacon which contains a fluorescence component (F) and a quencher (Q) in the figure. As illustrated the F and Q ends are in close proximity when the stem region is hybridized. When the molecular beacon is combined with the L-DNA, the loop read of the molecular beacon (labelled L-DNA in the figure) binds to the L-DNA and results in an increased distance between the fluorescent compound and the quencher. When exposed to a light with the excitation frequency of the F compound fluoresces unless near the quencher. This change produces an increase in fluorescence. In this design and this increase indicates the presence of an L-DNA that is a perfect match for the molecular beacon sequence that is applied during the decryption process.

FIG. 2. Strand hybridization fluorescence quenching detection. In this design, the encryption reagent is a specific sequence of L-DNA containing a quencher (Q) at a known base which is embedded in the test material during manufacture. During material testing the embedded material is retrieved from the material and mixed with the decryption reagent here an L-DNA strand containing a fluorescence tag (F) at a specific base of the sequence (shown here at one end) that aligns with the embedded sequence quencher. When exposed to a light with the excitation frequency of the F compound a fluorescence signal is detected unless the quenching compound is also present. As illustrated the F and Q ends are in close proximity if and only if they are both present (one from the material and one from the decipher process). In this design, the decrease in fluorescence indicates the presence of an embedded L-DNA that is a perfect match for the L-DNA sequence that is applied during the decryption process.

FIG. 3. Aptamer-Antibody Detection. In this design, a specific sequence of L-DNA (an aptamer) is used as the encryption reagent and embedded in the test material during manufacture. The aptamer is designed to specifically bind a region on a protein which is bindable by a specific antibody to that protein. During material testing the embedded aptamer is retrieved from the material and mixed with two other decryption reagents, a protein and an antibody to a specific region of the protein. In this design, the decrease in fluorescence indicates the presence of an embedded L-DNA aptamer that blocks protein-antibody binding during the decryption process.

FIG. 4. Microarray Detection. In this design, a specific sequence of L-DNA serves as the encryption reagent and is embedded in the test material during manufacture. The aptamer is designed to specifically bind a region on a microarray chip containing complementary L-DNA strands. The decryption component in this case is a microarray of L-DNA strands as potential binders. Detection of the binding is detected by traditional microarray detection methods to determine which region of the chip (if any) has been bound by the material retrieved for testing during the decryption process.

FIG. 5. Mirror-image restriction enzyme detection. In this design, a specific sequence of L-DNA containing a restriction site serves as the encryption reagent and is embedded in the test material during manufacture. During testing this embedded material is retrieved and mixed two other decryption reagents. The first of these is a molecular beacon design similar to that of FIG. 1 that also includes the complement to the restriction site of the embedded L-DNA. The second is a mirror-image restriction enzyme that is specific for the restriction site. When embedded L-DNA is present during the decryption process, the molecular beacon is cleaved by the restriction site and this produces an increases in fluorescence.

FIG. 6. Non-enzymatic L-DNA amplification. In this design a specific L-DNA sequence serves as the encryption reagent and is embedded in the test material during manufacture which when present is detected by one of several decryption methods based on non-enzymatic amplification methods. These include catalyzed hairpin assembly, branched hybridization chain reaction, dendritic amplification and dumbbell amplification. The components of the reactions consist of DNA sequences which are all constructed from L-DNA. The key to these designs is that they are amplification schemes that are triggered by the specific L-DNA sequence retrieved during the decryption testing.

FIG. 7. L-DNA enzymatic amplification by mirror-image polymerase and L-dNTPs. In this speculative design detects an encryption reagent, namely a specific L-DNA sequence embedded in the test material during manufacture by L-DNA specific polymerase amplification methods. A recent paper by Chen et al., Nature Biotechnol., doi.org/10.1038/s41587-022-01337-8 (2022) reports on the use of such a polymerase designated by the manufacturer as TransStart® PastPfu Fly DNA polymerase (TransGen Biotech Co., LTD, Beijing, China). The decryption reagents consist of the polymerase, forward and reverse primer components of the reaction consist of DNA sequences which are all constructed from L-DNA and L-dNTPs. The key to this design is that an amplification scheme analogous to existing DNA amplification is performed in a standard PCR reaction. Product is only produced if the specific L-DNA sequence retrieved during the decryption testing is present.

FIG. 8. L-DNAzyme detection. In this design the encryption reagent is a specific L-DNA lysozyme structure embedded in the test material during manufacture. This reagent has the property that it can act enzymatically to cleave a particular sequence of L-DNA which in this example serves as the decryption reagent. During testing a quenched fluorescent structure containing the cleavage target for the DNA-zyme is added to the material retrieved for testing. If the lysozyme made from L-DNA is present it will cleave the test material and create an increase in fluorescence.

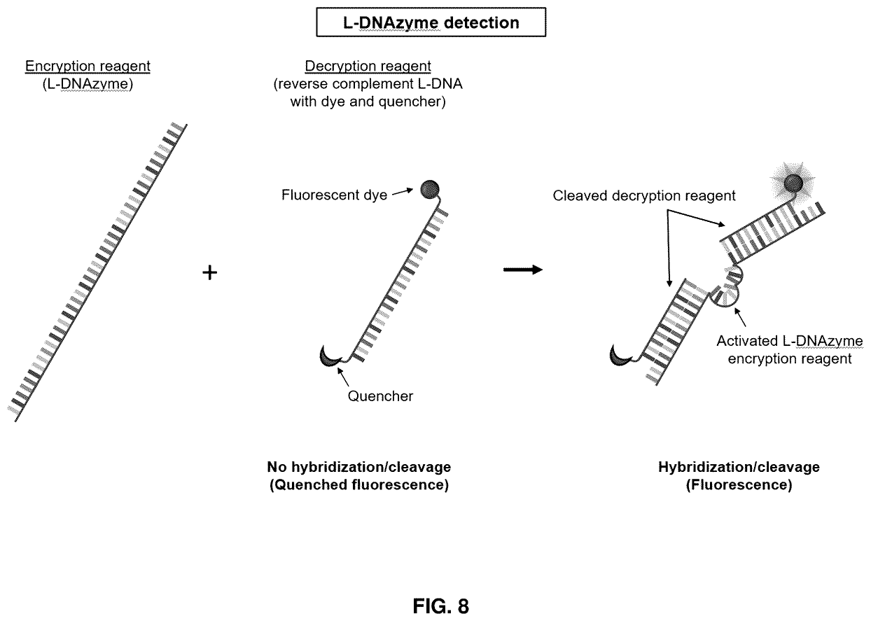

### DESCRIPTION OF ILLUSTRATIVE EMBODIMENTS

As discussed above, there is an impending need for new and creative methods and materials for labeling and tracking a variety of commercial/industrial/pharmaceutical process. The present inventors propose the use of left-handed DNA (L-DNA) tracers that can be used to identify the source, track the distribution, and validate the integrity of products or resources that are highly regulated, valuable, or hazardous (e.g., pharmaceuticals, treated water, chemicals, designer products, and ammunitions). As explained in greater detail below, L-DNA is the left-handed enantiomer (mirror image molecule) form of natural right-handed DNA (D-DNA). Similar to the way genetic information is encoded in natural DNA, the L-DNA tracers can be designed programmed to encrypt unique identifying information, as well as more general information about the type of product, such as the manufacturing location, source, and date, directly into the nucleotide sequence.

L-DNA tracers can be embedded directly into the product, such as a pill or explosive powder, so that it could neither be disassociated from the product nor be re-associated with another product. Because there are no technologies available to sequence L-DNA, the L-DNA tracers cannot be reverse engineered, copied, or falsified. The L-DNA tracers can only be deciphered using a unique detection key. L-DNA tracers therefore preserve the integrity of product supply chains, allow counterfeit products to be detected, and identify the source of highly regulated products.

L-DNA has a number of features that make it ideal as an imbedded tracer. First, as mentioned above the encryption of L-DNA tracers is secure. Because the technology to sequence L-DNA does not exist, the information stored in the L-DNA tracers cannot be determined, reverse engineered, or counterfeited. The presence of a unique L-DNA tracer can only be determined by the unique L-DNA detection key that is specific to each tracer sequence. Second, much like D-DNA, L-DNA can store an immense amount of information in a very small molecule. A single strand just 20 nucleotides (12.6 nanometers) long has nearly 1.1 trillion (4²⁰) unique sequence combinations. Third, L-DNA is biologically inert and resistant to degradation. There are no natural nucleases that target and degrade L-DNA. While the human physiological response to L-DNA has not been studied, it is recognized that L-DNA would survive substantially longer in the bloodstream and would be cleared from the body through urinary excretion (Williams et al., “Bioactive and nuclease-resistant L-DNA ligand of vasopressin” PNAS 94: 11285-11299,1997). And fourth, L-DNA is easily and inexpensively manufactured in large quantities. L-DNAs are synthesized using the same phosphoramidite chemistry that is used to synthesize DNA oligonucleotides.

These and other aspects of the disclosure are described in detail below.

### I. L-DNA

L-DNA is the enantiomer of the natural D-DNA. L-Deoxyribose, the sugar backbone of L-DNA, is mirror of natural D-deoxyribose and has not been found in nature despite the fact that other L-sugars (e.g., L-arabinose, L-lyxose, L-galactose, L-sorbose, and L-xylulose) do exist in nature. L-DNA can hybridize with complementary L-DNA sequences via classical Watson—Crick base-pairing, forming a L-DNA duplex much like a D-DNA duplex, except that the L-DNA duplex is a left-handed B-helix, a mirror structure of the right-handed A-helix by D-DNA. Importantly, however, L-DNA does not hybridize with the complementary sequence on D-DNA backbones or D-RNA at physiological temperatures. This unique base-pairing system of L-DNA has been exploited in molecular beacon technology for better target selectivity than D-DNA and as barcodes in DNA microarray to encode the DNA oligonucleotides without interfering the binding of targets.

Given that L-DNA is the enantiomer of D-DNA, the chemical reactivity of L-DNA is identical to D-DNA if the reactant is achiral. The biological reactivity of L-DNA with the chiral proteins and enzymes, however, is completely distinguishable from that of D-DNA. For example, L-DNA is not susceptible to natural DNA-modifying enzymes (e.g., ligases, polymerases and nucleases that can easily degrade natural D-DNA). Taking advantage of these properties, L-DNA has been used to build intracellular nano-sensors as well as aptamers exhibit an enhanced serum stability. L-DNA has also been employed to construct a self-assembled DNA tetrahedral nanostructure showing high cellular uptake. L-DNA has also been adapted to PCR (see Adams et al., Anal. Chem. 89(1):728-735, 2016) and used as a biostable DNAzyme with achiral metal ions (see Cui et al., Anal. Chem. 88: 1850-1855, 2016). Various aspects and uses of L-DNA is reviewed in Young et al., Chen. Eur. J., 25: 7981-7990 (2019).

### II. NUCLEIC ACID DESIGN

The present disclosure, in one aspect, is directed to unique configurations of L-DNA segments that can be used to mark or label a variety of subject matter, as further discussed below. Each nucleic acid molecule employed will comprise at least a first region comprising or consisting of an L-DNA segment comprising a randomized contiguous sequence that is a unique identifier for the nucleic acid molecule. Optionally, a second region comprising a non-randomized contiguous nucleic acid sequence that is correlated with at least one piece of data or information is included. In some embodiments, a third region may be included that comprises a capture moiety allowing the nucleic acid to be isolated away from its manufactured or “marked” environment.

The second and/or third regions may comprise or consist of an L-DNA segment, a D-DNA segment, or is a chimeric L-DNA/D-DNA segment. The first region must be an L-DNA segment. The second and third regions may be L-DNA segments and the first and second regions may be separated by the first DNA spacer and the second and third regions may be separated by a second DNA spacer. The first and/or second DNA spacers may be substrates for a nuclease. The length of the spacer may be about 0 to about 20 nucleotides. These are merely exemplary orders as the first, second and optionally third sequence can occur in any order, as shown below:
5′—First Region—Second Region—3′
5′—Second Region—First Region—3′
5′—First Region—Second Region—Third Region—3′
5′—First Region—Third Region—Second Region—3′
5′—Third Region—Second Region—First Region—3′
5′—Third Region—First Region—Second Region—3′
5′—Second Region—First Region—Third Region—3′
5′—Second Region—Third Region—First Region—3′

The nucleic acid molecule may be about 10 to 150 nucleotides in length. All that is required is that sufficient length be provided to encode a sufficient amount of information, i.e., the first random region and the second non-random region. In certain embodiment, the first region is 15 to 50, 15 to 30 or 15-20 nucleotides in length. In certain embodiments, the second region is 10 to 30 nucleotides in length. In certain embodiments, the third region is 10 to 30 nucleotides in length.

The second region may relate to data or information about the article, object or organism into which the L-DNA is placed. This could include one or more a date of manufacture, a location of manufacture, a manufacturer, a lot number, or an expiration date, such as for an associated product. Such information would be retained in a database correlated with the non-random sequence in the nucleic acid. The third region may comprise a capture sequence. The nucleic acid may also comprise a quenchable or activatable/inducible signaling agent.

### III. L-DNA SYNTHESIS

The first chemical synthesis of L-DNA oligonucleotides was reported by Anderson et al., Nucleoside, Nucleotides, Nucleic Acids, 3: 499 (1984) who prepared L-dU 18-mer by using the triester approach (Reese, Tetrahedron 34: 3143, 1978; Narang et al., Methods Enzymol. 65: 610, 1980). A more recent publication describes synthesis using either α-L pr f3-L nucleotide unnits and covalently linked to an acridine derivative (Asseline et al., Nucleic Acids Res. 19: 4067-4074, 1991).

### IV. ARTICLES OF MANUFACTURE/LABELING USES

L-DNA tracers as disclosed herein can overcome the challenge of reliably tracking the origin, distribution, and appropriate use of highly regulated, valuable, or hazardous products or resources. There are hundreds of potential applications for L-DNA tracers across many industries, including pharmaceutical tracking, drug compliance urine testing, currency authentication, treated water validation, upscale food or drink authentication, pesticides tracking, chemical tracking, designer product authentication, ammunition tracking, and explosive tracking.

Thus, in a broad sense, the material to be tagged or labeled is a chemical or biological composition. A chemical composition could a pharmaceutical grade chemical composition, a chemical herbicide, a chemical pesticide, a toxic or biohazard composition. A biological composition could be a pharmaceutical-grade biological composition, biological herbicide, a biological pesticide, or a biological warfare composition. The material may be is an article of manufacture, such as a pill or tablet, an explosive, a foodstuff or food additive, a cosmetic, a fuel, currency, an article of clothing such as a shoe, a shirt, a skirt, a dress, a coat, sunglasses, a purse or handbag, a passport or other legal document, an electronic device, a computer chip, or a vaccine. These are of course not limiting examples as the list of possible materials is essentially endless.

As discussed above, one particular example is the use of L-DNA tracers in tracking regulated pharmaceutical products. Falsified medicines for all kinds of illnesses, including cancer, heart disease, asthma, and mental health disorders, have been reported and the application of this technology to prevent the introduction of counterfeit prescriptions from entering into the supply chain. L-DNA tracers will complement this system and improve the security of the distribution channels. Instead of relying solely on the barcoded packaging, by adding L-DNA tracers directly into the formulations of individual medicines, the manufacturers, distributers, and pharmacists will be able to test the products directly. The inventors envision a simple test where a sample of the medicine is mixed into a testing buffer containing the relevant L-DNA key and the validity of the medicine would be validated by a positive result.

### V. DETECTION METHODS

There are two aspects to the detection process for the L-DNA tracers described herein. The first is to simply capture the L-DNA such that its presence (or absence) can be confirmed in any particular product, article or environment. The second would involve “decoding” the information in the L-DNA once it was determined that it exists. This involves the use of a unique L-DNA key that can bind to and hence identify the sequence found in the second region.

### VI. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—DNA Ladder Labeling

A DNA ladder can be validated/identified based on an L-DNA included in the product as single stranded encryption reagent with an L-DNA sequence length within the range of the product ladder. To validate the ladder in a gel, the user adds an L-DNA ‘key’ reagent before use that binds through sequence homology to the L-DNA already in the product to produce a unique component viewable during gel visualization. The presence of the matched encryption-decryption pair authenticates/validates the ladder's origin.

### Example 2—Prophetic Examples

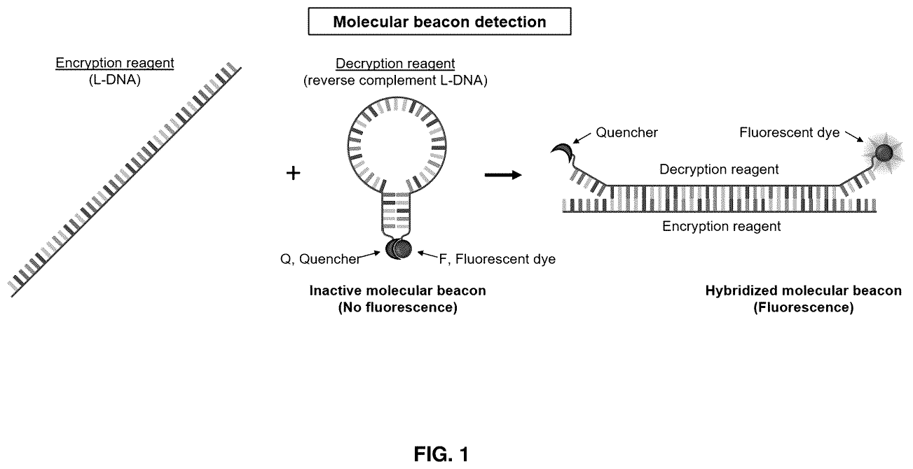

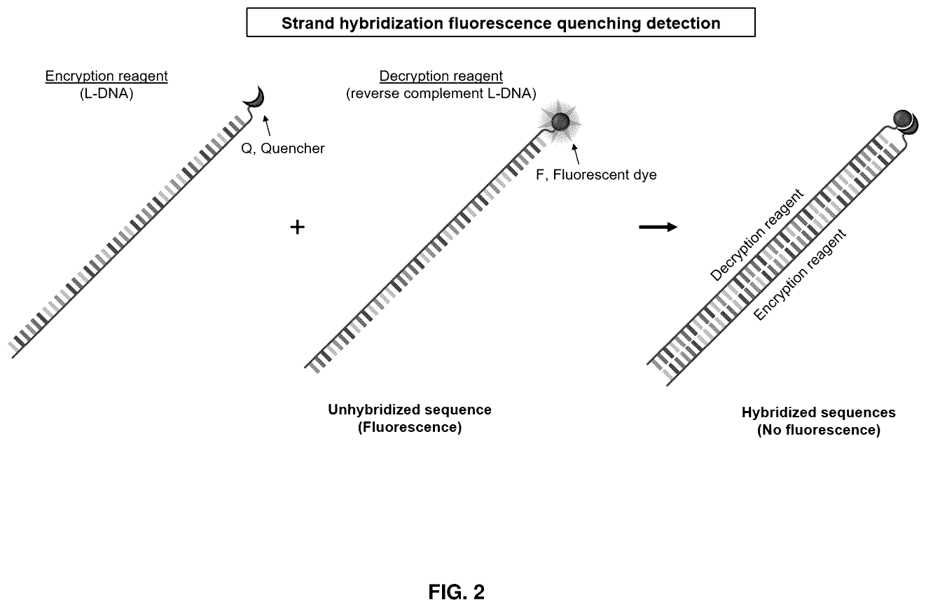

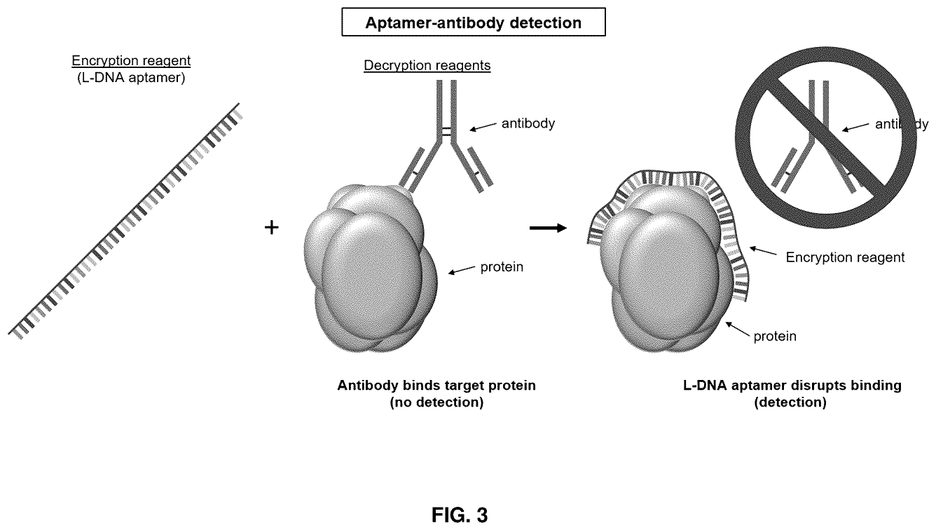

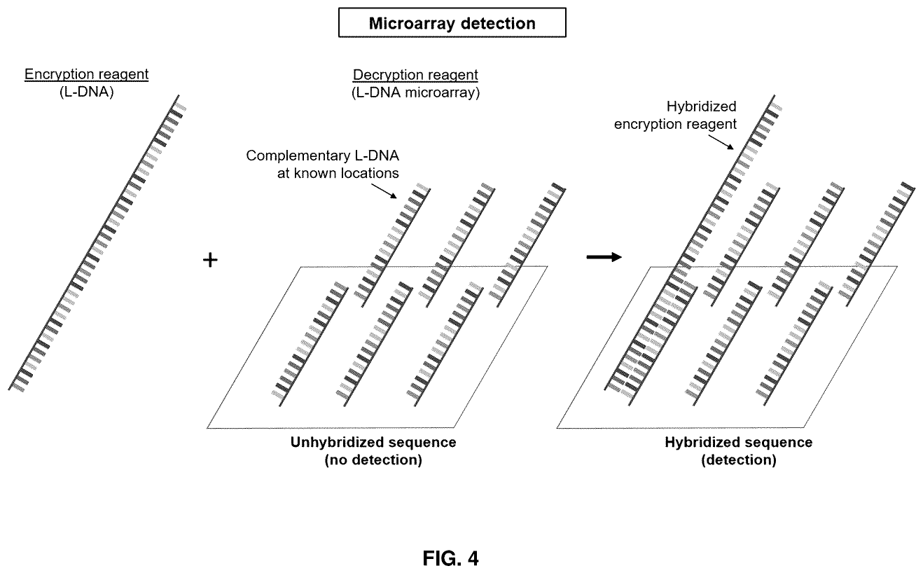

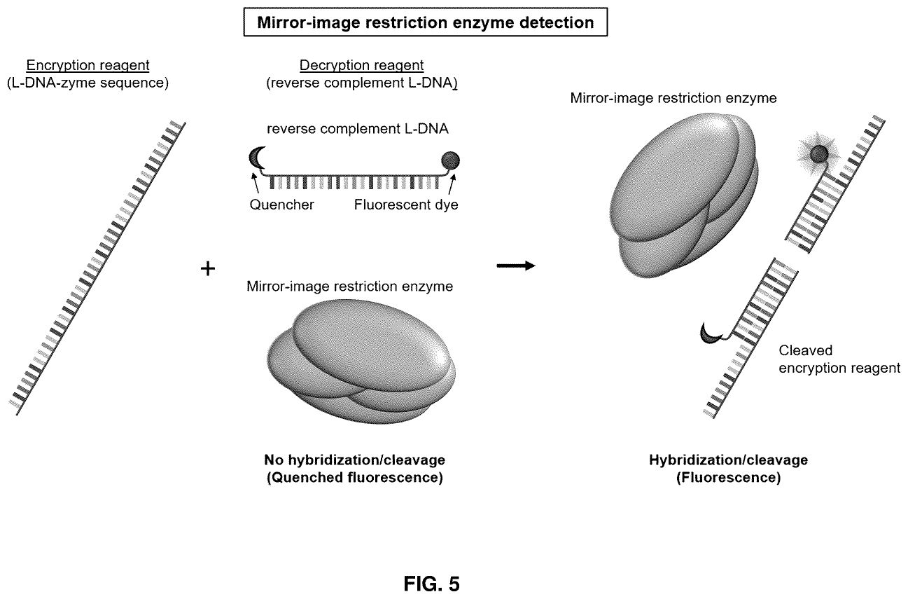

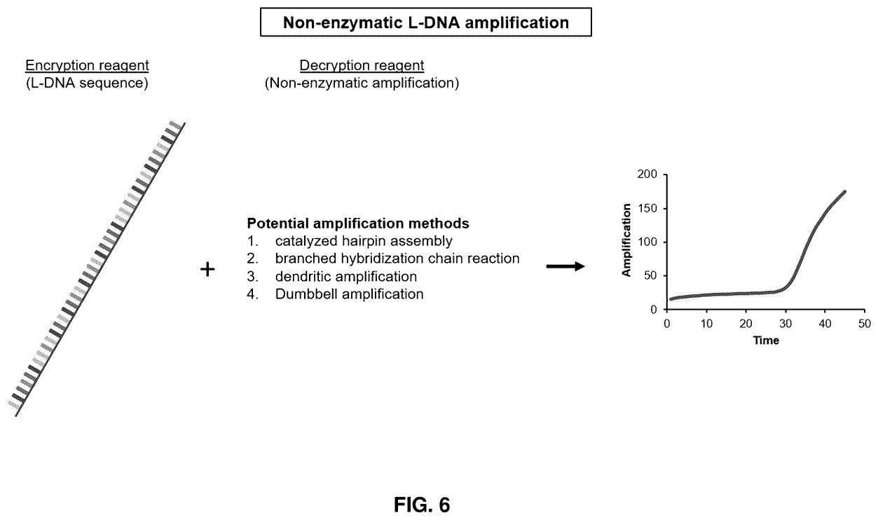

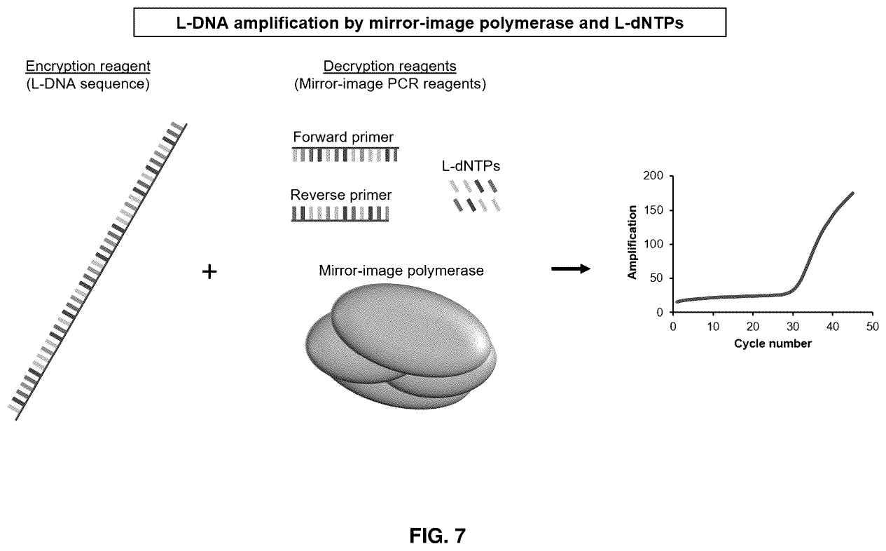

Product Validation. There are several possible examples envisioned here. Some examples include a methods to detect counterfeit products. For example, to validate that a pharmaceutical, an expensive fashion item, or currency is not counterfeit. In these cases, an L-DNA compound can be incorporated into a product as an encryption key and this compound can be verified as present only by the decryption test. The decryption cannot be bypassed without knowing the initial compound added to the product. For instance, to verify that a pill has been manufactured and certified by a producer, a short sequence of known L-DNA, such GCAGCTTGCAATTGGACGG (SEQ ID NO: 1), can be synthesized and added directly to the product or provided as part of a product insert. If a question is raised about the validation, methods to isolate DNA can be employed to recover the DNA and one of the decryption methods outlined in FIGS. 1 to 7 can be used to verify the product's authenticity.

Pharmaceutical use validation or drug compliance monitoring. A second key property here with respect to the biology and potential applications is that L-DNA structures are completely inert biologically. The use of a prescribed pill could then be monitored since when they are ingested the L-DNA is not broken down and the L-DNA tag present in the pill would not be broken down by normal biological processes. Therefore, these structures would be present in urine for instance or in stool or in the blood. The detection of the L-DNA tracer would allow one to know that a pill containing an encryption code was recently ingested. One particular example that is well-known is in the monitoring of treatment for some tuberculosis drugs which require long-term (like consistent ingestion of a prescription on a daily basis). The current implementation is dependent on tracking monitoring by directly observing that the recipient takes the drug in the presence of the provider, sometimes over a 6-month period.

Hidden tracking applications. A third broad application might be as tracers to track the origin of materials like explosives or crops. An example would be adding a tracer to nitrate compounds that are sometimes co-opted as explosives. Even after an explosion, DNA structures would be expected to survive so that an incorporated L-DNA that was associated with a particular fertilizer batch during production could be used to track and examine how the materials were obtained. Crop validation is another area where an L-DNA tag could be added during crop maintenance (like pesticide spraying) and then be used to trace the crop without compromising the crop as a food source. This application is also enabled by the biologically inert nature of the L-DNA. In this application, one can imagine this tool might be of use in import validation and control.

Laboratory reagent validation. There are some reagents that are used in testing and require a validation step, i.e., to certify molecular reagents. The key to this approach is similar in that it includes embedded structures and includes a known detection key to validate their origin of manufacture and possibly some aspects of its performance.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

1. A nucleic acid comprising a first region that is an L-DNA segment having a randomized contiguous sequence that is a unique identifier for the nucleic acid molecule.

2. The nucleic acid of claim 1, further comprising:
(a) a second region comprising a non-randomized contiguous nucleic acid sequence that is correlated with at least one piece of data or information associated with said product; and/or
(b) a third region comprising a capture moiety.

3. The nucleic acid of claim 1, wherein the first region is at least 10 nucleotides in length, at least 15 nucleotides in length, at least 20 nucleotides in length, at least 25 nucleotides in length, or 10 to 30 nucleotides in length.

4. The nucleic acid of claim 2, wherein the second region is at least 5 nucleotides in length, at least 10 nucleotides in length, at least 15 nucleotides in length, at least 20 nucleotides in length, at least 25 nucleotides in length, or 10 to 30 nucleotides in length.

5. The nucleic acid of claim 2, wherein the third region is at least 5 nucleotides in length, at least 10 nucleotides in length, at least 15 nucleotides in length, at least 20 nucleotides in length, at least 25 nucleotides in length, or 10 to 30 nucleotides in length.

6. The nucleic acid of claim 1, wherein the nucleic acid is about 10 to 150 nucleotides in length, about 20 to 120 nucleotides in length, about 20 to 100 nucleotides in length, about 25 to 75 nucleotides in length, 15 to 50 nucleotides in length, 15 to 30 nucleotides in length, 15 to 20 nucleotides in length, or about 30 to 50 nucleotides in length.

7. The nucleic acid of claim 1, wherein the at least one piece of data or information comprises a date of manufacture, such as for an associated product, a location of manufacture, such as for an associated product, a manufacturer, such as for an associated product, a lot number, such as for an associated product, an expiration date, such as for an associated product.

8. The nucleic acid of claim 2, wherein said second region comprises or consists of an L-DNA segment, a D-DNA segment, or is a chimeric L-DNA/D-DNA segment.

9. The nucleic acid of claim 2, wherein said third region comprises or consists of an L-DNA segment, a D-DNA segment, or is a chimeric L-DNA/D-DNA segment.

10. The nucleic acid of claim 2, wherein said first and second regions are L-DNA segments and are separated by a first D-DNA spacer.

11. The nucleic acid of claim 2, wherein said first, second and third regions are L-DNA segments and said first and second regions are separated by the first DNA spacer and said second and third regions are separated by a second DNA spacer.

12. The nucleic acid of claim 10, wherein said first and/or second DNA spacers are substrates for a nuclease.

13. The nucleic acid of claim 2, wherein said third region comprises a capture sequence.

14. The nucleic acid of claim 1, wherein said nucleic acid comprises a quenchable or activatable/inducible signaling agent.

15. The nucleic acid of claim 2, wherein said first, second and optionally third sequence can occur in any order.

16. A method of labeling a product comprising embedding in or attaching to a product a nucleic acid according to claim 1 (“tracer”) comprising a first region that is an L-DNA segment having a randomized contiguous sequence that is a unique identifier for the nucleic acid molecule.

17. The method of claim 16, wherein the product is a chemical composition, such as a pharmaceutical grade chemical composition, a chemical herbicide, a chemical pesticide, a toxic or biohazard composition.

18. (canceled)

19. The method of claim 16, wherein the product is a biological composition. such as a pharmaceutical-grade biological composition, biological herbicide, a biological pesticide, or a biological warfare composition.

20. The method of claim 16, wherein the product is an article of manufacture, such as an article of manufacture is a pill or tablet, an explosive, a foodstuff or food additive, a cosmetic, a fuel, currency, a passport or other legal document, an electronic device, a computer chip, or a vaccine.

21.-25. (canceled)

26. A method of identifying a product or sample comprising contacting an L-DNA “key” molecule with said product and determining hybridization of said L-DNA “key” to a complementary L-DNA tracer in said product, the present of said complementary L-DNA “label” being correlated with one or more identifying features for said product.

27.-35. (canceled)


SEQUENCE LISTING (1 sequence)

SEQ ID NO: 1 - DNA, 19 bp, synthetic construct
  source 1..19: mol_type = other DNA
gcagcttgcaattggacgg